(19) United States
(12) Patent Application Publication
(10) Pub. No.: US 2023/0272322 A1
(43) Pub. Date: Aug. 31, 2023

(54) BREWING SYSTEM, BIOREACTOR PROVIDED WITH SUCH A SYSTEM AND IMPLEMENTATION METHOD THEREOF

(71) Applicant: JUA, MONTPELLIER CEDEX 2 (FR)

(72) Inventor: Jean-Luc SALLUSTRO, TROU-AUX-BICHES (MU)

(73) Assignee: JUA, MONTPELLIER CEDEX 2 (FR)

(21) Appl. No.: 18/005,602

(22) PCT Filed: Jul. 13, 2021
(86) PCT No.: PCT/IB2021/056283

§ 371 Date: Jan. 14, 2023

(30) Foreign Application Priority Data
Jul. 16, 2020 (FR) FR2007481

(51) Int. Cl. C12M 1/00; C12M 1/02; C12M 1/26; C12M 1/34

(52) U.S. Cl. CPC C12M 23/26; C12M 41/18; C12M 33/04; C12M 23/58; C12M 41/44

(57) ABSTRACT

Said brewing system is intended to be fitted to a bioreactor which can be used in particular in milk and cheese factories, fermented dough production units, breweries, wine-making units or even low-temperature fermentation units in an aerobic or anaerobic environment, in particular lacto-fermentation units for the purpose of preserving vegetables, wastewater treatment stations, fish farming ponds with or without temperature control. According to the invention, said brewing system comprises:—at least one flexible and sealed brewing chamber (2, 3),—means (4) for receiving a fluid referred to as the filling fluid,—means (5, 51, 70, 71) for transferring the filling fluid between the brewing chambers (2, 3), and means (4) for receiving the filling fluid as well as varying the volume of the filling fluid admitted into each brewing chamber.

### CROSS-REFERENCE TO RELATED APPLICATION

The present application is a National Stage Application of PCT International Application No. PCT/IB2021/056283 (filed on Jul. 13, 2021), under 35 U.S.C. § 371, which claims priority to French Patent Application No. FR 2007481 (filed on Jul. 16, 2020), which are each hereby incorporated by reference in their complete respective entireties.

### TECHNICAL FIELD

The present invention relates to a slow brewing method with non-destructive positive displacement for a more or less viscous fluid environment that also optionally ensures the role of heat carrier and favours the installation and the maintaining of a bioturbation zone in a bioreactor.

### BACKGROUND

Below, the modes of brewing and of heating, as well as of biological activation of the bioreactors and fermenters, of the aerobic or anaerobic type, will first be presented.

Bioreactors are artificial ecosystems that install, protect, favour and maintain a targeted biological activity, most often microbial, that is to say in the presence of bacterial flora, yeast or even both, in a partly or totally closed chamber. The fluids contained therein with a biochemical trophism effect can be more or less viscous, either by nature or because of their development in the bioreactor, but in the large majority of cases they must be brewed with a homogenisation effect to allow for example:
the vertical and horizontal mobilisation of flocculated particles at the sedimentary horizon in order to intensify or perfect the trophisms such as biooxidation,
the selective sedimentation of inert particles in a dedicated zone or their regular evacuation can be favoured,
in general, the creation of slow turbulences of the swell type at medium amplitude and low frequency,
a homogenous diffusion of heat in the substrate,
homogenous and rapid or intense exchanges between the substrate and the bacterial flora, the biological reactants, the enzymes and coenzymes,
the diffusion of biogenic or exogenous gases in the substrate,
the solid/liquid phase separation, and
the prevention of inhibiting coalescences such as the formation of floating crusts or of solid sediments.

The brewing capacity of hydraulic devices, such as pumps ensuring a powerful recirculation of the substrates, or mechanical devices such as plates, propellers or rotary screens, should not be questioned in terms of the structural efficiency of the brewing. However when the microbial biomes undergo their effects, the assemblies in nodules or in clusters that form without exception in a biologically active environment are partly destroyed, which certainly statistically or geometrically increases the diffusion of the microbes in a given volume but also proportionally reduces their vitality. Indeed, it is accepted that the bacterial architectures form on a minimal base of commensalisms and more so to support functional symbioses, likewise the yeast requires the presence of enzymes in osmotic position, however the reconstruction of these assemblies requires energy and materials, generally biofilms, and these metabolic costs are incurred to the detriment of the desired trophism on the substrate.

With regard to the heating, it is also very routine for these substrates to require a precise, progressive and non-destructive thermal regulation which is most often obtained with a system of the bain-marie type or by injection of a neutral solvent at critical temperature (water for example). In these two cases the thermal gradient between the heating source in contact or in intrusion and the substrate to be heated must be weak. For this the mass of the heat transfer fluid and the exchange surfaces must be relatively large, much greater for example than with an electric immersion heater. It is indeed well known that high temperatures, generally above 70° C. are harmful to the bacterial flora however in the zones of contact of an electric immersion heater or of coils supplied with vapour this temperature is quickly reached and exceeded with the effect of definitive damage to the biotic parameters and even to the nature of the substrate (for example MAILLARD reaction).

There is a large number of agro-industries, at which the invention can be aimed. The latter use methods that must combine the parameters presented above, relative to the heating and brewing parameters respecting the microbial biomes and the yeast. These are in particular milk and cheese factories, fermented dough production, breweries, wine-making and by extension all the low-temperature fermentation units in an aerobic or anaerobic environment, in particular lacto-fermentations for the purpose of preserving vegetables.

The combination of pulsed brewing with non-destructive positive displacement and precise inertial heating with a weak gradient is particularly required in mesophilic or thermophilic anaerobic methane digesters as explained below.

The methods for treating wastewater will also advantageously benefit from this method to create with or without the addition of heat a slow swell effect at tank bottom in systems with granular sludge or simply to intensify the micro-oxygenation effect with an effect of aerobic biooxidation.

The combination of pulsed brewing with non-destructive positive displacement with a swell effect and precise inertial heating with a weak gradient is particularly required in mesophilic or thermophilic anaerobic methane digesters as explained below.

Indeed, the treatment of organic matter by biodigestion is first and foremost subjected to biological constraints that attempt to respect the techniques that aim to create and maintain an ecosystem favourable to the microorganisms specific to this type of biooxidation. To simplify, it can be said of a biodigester that it receives and maintains in a bioreactor populations of strictly anaerobic microbes that are led to grow and reproduce on an organic substrate consisting of liquid or solid matter placed in solution. In essence these specific microbial populations develop an activity of biooxidation, but in the absence of the oxygen of the air. The reaction is only possible when the three bacterial communities typical of this trophism constitute a balanced ecosystem so that most of the reducing equivalents (atoms of carbon and of hydrogen) produced as waste during the bacterial anabolism (hydrolysis then acidophilic and acetogenesis) finally end up in the methane (methanogenesis). The bacterial species in question are complex and relatively varied but their biochemical characteristics and the main principles of their ecology are rather well known. They are generally classified into three groups:
the hydrolytic and fermenting bacteria,
the acetogenic bacteria, and
the methanogenic bacteria.

The management of the artificial ecosystem formed by an anaerobic bioreactor requires dynamic intervention to ensure certain essential physico-chemical conditions, such as the pH, the temperature and the oxidoreduction potential and the nutritional needs. The availability of digestible carbon is in particular critical to avoid fatal inhibitions in the presence of volatile fatty acids or of supernumerary ammonium and to optimise the production of methane.

The optimal pH of the anaerobic digestion is located around neutral. It is the result of the optimal pH of each bacterial population: that of the acidifying bacteria is located between 5.5 and 6, the acetogenic prefer a pH close to neutral while the methanogenic have a maximum activity in a pH range between 6 and 8. However, anaerobic digestion can occur in slightly acidic or basic environments.

The activity of the methanogenic consortium is closely linked to the temperature. Two ranges of optimal temperatures can be defined: the mesophilic zone (between 35° C. and 38° C.) and the thermophilic zone (between 55° C. and 60° C.) with a decrease in the activity on either side of these temperatures. The majority of the bacterial species were isolated in mesophilic environments, but all the trophic groups of the steps of anaerobic digestion have thermophilic species using the same metabolic pathways as the mesophilic bacteria with analogous or greater performance. It remains nevertheless possible to work at temperatures different from the optima with lesser performance.

With regard to the oxidoreduction potential, this parameter represents the state of reduction of the system, it affects the activity of the methanogenic bacteria. These bacteria indeed require, besides the absence of oxygen, an oxidoreduction potential lower than 330 mV to initiate their growth.

With regard to the nutritional and metabolic needs, like any microorganism, each bacterium forming the methanogenic flora requires a sufficient supply of macronutrients (C, N, P, S) and of micronutrients for their growth. The needs in terms of macronutrients can be roughly evaluated on the basis of the raw formula describing the composition of a cell (C₅H₉O₃N). For the methanogenic bacteria, the culture medium must have concentrations of carbon (expressed as Chemical Oxygen Demand (COD)), of nitrogen and of phosphorus at minimum in the COD/N/P proportions equal to 400/7/1.

Ammonium is their principal source of nitrogen. Certain species capture molecular nitrogen while others need amino acids. The needs in terms of nitrogen represent 11% of the volatile dry mass of the biomass and the needs in terms of phosphorus 1/5 of those of nitrogen.

The methanogenic bacteria have high concentrations of Fe-S proteins which play an important role in the electron transport system and in the synthesis of coenzymes. Thus, the optimal concentration of sulphur varies from 1 to 2 mM (mmol/L) in the cell. This flora generally uses the reduced forms like hydrogen sulphide. The methanogens assimilate phosphorus in mineral forms.

Certain micronutrients are necessary for the growth of the methanogens. These are more particularly nickel, iron and cobalt. Indeed, they are components of coenzymes and of proteins involved in their metabolism. Magnesium is essential since it comes into play in the terminal reaction of synthesis of the methane as well as the sodium appearing in the chemico-osmotic process of synthesis of adenosine triphosphate (ATP).

There are growth factors stimulating the activity of certain methanogens: fatty acids, vitamins and complex mixtures such as yeast extract or trypticase peptone.

In conclusion, although today the “macro-model” that simulates a biodigestion process is correctly mastered, to the point that the extent and the form of methane production and of the composition of a digestate can be summarily predicted, the methods are nevertheless difficult to implement. Indeed, if it is desired to treat a given organic effluent, the fermentable fraction of household waste and equivalent or certain organic waste industrial or coming from agricultural sectors, or a mixture of intrants (co-digestion), each time the process must be dedicated to achieve the best productivity since to each substrate corresponds an optimal microbiological ecosystem and the biochemical yield ranges are narrow. In other words, the technical goal involves designing and implementing a methane digester with low investment and low operating costs but perfectly versatile in terms of bacterial resources to ensure high methane productivity regardless of the variation of the constraints of intrants.

Various methods are capable of allowing the implementation of a bioreactor. In general it is meant that a bioreactor under anaerobic conditions is an artefact that attempts to optimise the living conditions of a colony of given microorganisms at a given time and/or in a given location in order to concentrate in a minimal biological retention time, thus in a minimal bioreactor volume, the maximal production of methane that results from the digestion of the substrates placed in aqueous solution or more generally in the absence of gaseous oxygen. To simplify it can be said that a biodigester consists of four major components:
a sealed and often heat insulated chamber,
a stirring or brewing device,
a device for heating the digestates, and
input output devices for the substrate, the digestate and the biogas.

According to the methods implemented two major types of ecosystems are distinguished:
fixed biomass, and
free biomass.

In a fixed biomass digester, the chamber is used not only to contain the substrate and isolate it from the air but also to fix anaerobic bacterial colonies on suitable supports. Certain liquid phase techniques use independent fixing cells that are submerged in the flow. In general, the advantage of this method lies in the maintaining of the availability of the bacterial strains despite the permanent or sequential transfer of the flows of treated substrates, the desired goal being to not have to restart a bacterial inoculation or to avoid specialising the flora with chemical additions. Several types of fixing methods are available, some for example granulate the substrate or a part of the entering substrate before inoculating it and circulating it in the chamber of the biodigester.

As a general rule, the operations of biooxidation of waste or organic matter must satisfy several criteria of efficiency and of biosecurity that are performed by carrying out critical regulation and adjustments. Thus, in a digester with free or fixed biomass methods for reinforcing the active biomass are used that are the result of the reheating and of the circulation of the juices and optionally of additions of micronutrients and pH correctors. The method is adaptive and counts on the spontaneous capacity of the bacterial flora to specialise according to the constraints of the environment, in particular with regard to the presence of nutrients in a significant quantity. The adaptability of the biomass, left free to leave the chamber with the sequential or continuous flow of the flows, and to change according to the constraints of the ecosystem is reinforced by “outside” actions, thermal (maintaining in mesophilic at 36° C. or thermophilic 55° C. conditions), chemical (neutralisation of the acidic or basic pHs) and mechanical (transfers, fluidification and brewing). In general a biodigester thus requires either good monitoring of the indications provided by sensors, in order to allow a human response of adjustment in delayed time, or the analysis and the automatic processing of the signals transmitted by sensors inferring in real time the actuation of effectors.

Beyond the differentiation between fixed biomass and free population, manual or automated adjustments, two types of dynamics of the flows are also distinguished, with systems with continuous or sequential feeding:
sequential loading, and
continuous feeding.

The methods with sequential loading have the major characteristic that they seek to establish, in the same chamber for a single dose of substrate, the succession of the major phases of the methane digestion. In other words, it can be considered than in this context the bacterial populations develop on an identical substrate from the beginning to the end of the cycle and thus do not need to spend energy to adapt to unexpected changes in their ecosystem, it is they who transform it and not the opposite. Thus as soon as the loading of the tank is completed, and it can be carried out in a day or in three or four, the optimal conditions for starting the hydrolysis phase are provided (temperature, pH, nutrients, inoculation). Then comes the transient acidogenesis phase which is regulated to allow the triggering of the acetogenesis and finally of the methanogenesis. In theory this method is of interest because it has a Hydraulic Retention Time (HRT) shorter than that of the continuous-flow protocols and is easier to master. In general, several tanks operating in parallel that are activated one after the other as they are filled must be available. In case of malfunction of a cell the treatment can be continued with the others. It is also a method in which the tanks are smaller and which generally accept substrates denser in dry matter. Nevertheless, the sequential loading requires multiplying the chambers and the auxiliary devices such as the loading hoppers, the valves and other pumps.

Continuous feeding is strictly opposite to the sequential loading on several levels. First of all because the ecosystem and particularly the bacterial flora are led to be polyvalent, or rather to make the bacteria and their coenzymes coexist in the same chamber and at the same time but not necessarily in the same zone of the bioreaction volume for the four phases of the cycle. Then because to obtain a sufficient HRT the tank must be dimensioned for very large volumes which leads to proportional energy costs to maintain a suitable temperature (very rarely thermophilic except for small units) and specially to brew the mixture continuously in order to avoid the formation of a crust on the surface and sediments that are too dense at the bottom of the tank. It should be noted however that this method, very old since Chinese farm or household biodigesters are for the most part fed continuously, adapts well to microdeposits of homogenous organic substrates with very low variability. Indeed with very small dimensions (several tens of m3), waste of stable quality and quantity, they are easy to maintain supposing that it is not sought to evacuate the sediments in real time but rather the flows in liquid or turbid (eluates) phase that can then be used for spreading. Nevertheless after several cycles of operation these small units must be stopped and emptied of their sediments which by accumulating reduce the useful volume of the facility and have a negative effect on the development of the bacterial flora. Only certain industrial methods manage to produce, in addition to the biogas, highly loaded flows from which digestates that are generally difficult to use as biological fertiliser are extracted by decantation and/or pressing. The advantage of this method, at the industrial or domestic level, thus lies substantially in its capacity to accept a continuous flow of waste or of effluents with a low organic load with average production of biogas but possible reuse of the extracted effluents and with more difficulty of the “solid” fraction of the digestates.

On the basis of that which has just been described, two major types of methods continue to compete with each other, the infinitely mixed and the separation of phases:
single phase, and
differentiated phases.

In the first case, whether the biodigester is of the sequential or continuous type, with fixed or free biomass, all of the phases occur in the same chamber. This subsystem is either gravitational (sedimentation) or counter-flow and the vast majority are this type of digester. The fundamental technological variations relate to the modes of the sequential or linear mixing of the substrates (brewed versus pulsed versus infinitely mixed), the modes of introducing the substrates and of extraction of the digestates and eluates.

In the second case and in theory, each of the four phases can be confined in a distinct tank and the passage of the modified substrate after each phase to the following is ensured by a mechanical or hydraulic system. In reality the prior art clearly favours the two-phase systems in which hydrolysis and acidogenesis are confined in a first chamber while the acetogenesis and the methanogenesis are ensured together in the second chamber. The goal sought by these multiphase methods is to better manage the phases individually by playing with the micro-conditions optimising these various ecosystems. More complex and costly, the methods with differentiated phases nevertheless have better yield in terms of biodegradability in particular for substrates that require strong enzymatic speciation and/or a specific chemical or thermal environment. However for a flow of waste homogenous over time and having a composition that does not provide particular risks (especially in the acetogenesis stage) it is generally considered that this method does not provide sufficient added value to justify the complexity and the required investment.

Finally, the distinction is made between three types of biodigesters according to the concentration of Total Solids placed in Suspension (TSS) in the flows, that is to say the proportion of dry matter (DM) placed in solution in the digester:
low concentration of DM with less than 10% TSS,
medium concentration of DM containing between 10% and 20% TSS, and
high concentration of DM containing more than 20% TSS.

The applications of the principle of the biodigestion of flows with a low concentration of TSS have as the main intrants industrial or domestic effluents, as is the case for wastewater treatment stations. The biodigesters that are applied to treating this flow have a specific configuration, the principle involves using the biodigester as a sedimentation tank in which the TSS are retained and treated anaerobically while a flow of purified water exits. To put it more clearly, the Biological Retention Time (BRT) of the TSS is greater therein than that of the entering flow (HRT) since the biodigester integrates a passive or active decantation system and a system for anaerobic retention/degradation of the digestible DM.

As such these biodigesters are not suitable for the treatment of solid organic waste unless the latter are ground and placed in solution with effluents that always form the majority of the intrant. Under these protocols, the production of biogas and of methanocompost (in this case in the form of sludge) is relatively low but their capacity for primary purification of an effluent is very good and their energy balance is balanced with the cogeneration of the biogas. Optionally the productivity of these subsystems is improved with the resale of the liquors of digestates (eluates) as liquid organic fertilisers. The applicable maximum load volumes are approximately 2 to 5 kg of COD/m3/d.

The type of biodigester with a medium concentration of TSS is the most common, in this configuration a solid digestible substrate is placed in solution in 2 times to 3 times its weight in water. This mode of density of organic matter placed in solution corresponds to a search for balance between the quantity of digestible matter, its viscosity and its coalescence in the chamber of the digester and the capacity of the anaerobic environment to house and maintain bacterial populations without risking their inhibition via biochemical saturation. Indeed, for the bacterial activity to occur in the best conditions it is necessary for the digestate to not be compacted as long as it can be mobilised over the various phases of the biodigestion. This method thus adapts to the treatment of the digestible fraction of solid organic waste subject to an effective sorting upstream to evacuate the unwanted and a relatively fine grinding that allows the hydraulic transfer of the digestible mass and the proliferation of a strong bacterial diversity. More suitable for the methods with continual loading than sequential the principle of the medium concentration of DM takes advantage particularly of the systems with fixed biomass since the flow of substrate has a flow rate sufficiently high to weaken the resident flora. In general, the load volumes to be applied can reach 15 to 20kg COD/m³/d. The hydraulic residence times vary between 4 and 5 weeks. In this configuration the biogas yields are good and the production of methanocompost in the form of sedimented fibred matter is acceptable but requires at least a decantation if not a centrifugation.

Certain deposits of organic waste consist of a significant solid fraction with low digestibility. To put it simply the mass of DM is significant but the proportion of Volatile Organic Matter (VOM) to the DM is not high. Insofar as it is not possible to reasonably concentrate the VOM of this waste it is expedient to have available a technology that allows their treatment anaerobically and certain biodigesters are designed for this type of use. They are referred to as with a high concentration of DM.

The specificity of these uses lies in the mode of advancing and of brewing of the substrate and in the fact that these are almost exclusively bioreactors with sequential loading and free biomass, but with inoculation. In general, it should be noted that beyond a certain threshold of VOM content, there is a risk of overloading that can lead to an inhibition of the methanogenesis which is especially valid for the waste rich in animal proteins (carcasses and fats). Moreover, the load volumes to be applied can reach 40 kg COD/m³/d. The hydraulic residence times vary between 2 and 3 weeks.

The fact should thus be taken into account that beyond 3 g/L, ammonium (NH₄⁺) is an inhibitor of methanogenesis. It is also known that this limit of 3 g/L of NH₄⁺ must not be exceeded for waste, the C/N ratio of which is equal to or less than 20 with a concentration of VOM of approximately 60% of the OM.

The technique most used to maintain the organic substrates below this threshold involves mixing the waste too rich in proteins (viscera, fish, dairy products, carcasses and other meat waste) with carbon substrates. The alternative to the approach by regulation of the mixture involves lowering the concentration of VOM of the waste (especially the proportion of ammonium) by subjecting it to a previous phase of intense thermophilic aerobic fermentation, but this requires in any case the meat waste to be mixed with carbon substrates.

In conclusion, regardless of the methane digestion method chosen, three parameters strongly contribute to ensuring a satisfactory biological productivity and a recognised economic feasibility:
the non-destructive positive-displacement brewing of the digestates undergoing anaerobic maturation in the presence of bacterial colonies and their commensals and enzymatic resources which is particularly achieved when a zone of active bioturbation is maintained in the digester,
the treatment of the digestates to make them usable without environmental or biological risk as a biofertiliser, which is generally completed after liquid/solid phase separation and thermophilic aerobic maturation, and
the availability of digestible organic carbon in a progressively mobilisable form in order to always remain below an inhibition threshold of VFA and ammonium.

Among the known prior art, first of all, French Patent Publication No. 2 530 486 is cited. The latter describes the immersion of a flexible membrane in a fluid, which membrane has openings allowing the passage of the fluid from one side to the other. This solution does not allow to provide a satisfactory response to the issues presented above.

The Chinese Patent Publication No. 101 787 344 is also cited, which teaches submerging an envelope in a reactor, then varying the volume thereof to stir the reaction medium. However the teaching of this document is imperfect in the sense that the solution, which is presented therein, is described in reference to drawings which are only of the schematic type. Consequently, a person skilled in the art is not capable of deducing therefrom structural arrangements viable on the industrial level. In any case, the solution presented in this document is not capable of providing technical advantages to the reactors, the designs of which are significantly different from each other.

That being specified, the invention aims to overcome the disadvantages of the prior art as presented above.

The invention aims in particular to propose a brewing system for a bioreactor, the contribution of which in terms of biological productivity is significant while having a relatively simple mechanical structure as well as a convenient implementation.

The invention also aims to propose such a brewing system capable of adapting to various structural designs of this bioreactor and of the reaction species that it contains, in particular by favouring the fluidity of the tank-bottom sediments in order to facilitate their extraction by pumping.

The invention also endeavours to propose a brewing system, the swell effect of which can be easily regulated in amplitude and in frequency by a person skilled in the art, by acting in particular on hydraulic devices outside of the bioreactor.

The invention also aims to propose such a system, the brewing effect of which is accompanied by significant technical effect, on the thermal level.

Finally, the invention also aims to propose such a brewing system, the price of which is not very high, in particular because it is composed of routinely available mechanical elements.

### SUMMARY

At least one of the above goals is achieved via a brewing system intended to be provided in particular in a bioreactor implemented in particular in milk and cheese factories, fermented dough production units, breweries, wine-making units or even low-temperature fermentation units in an aerobic or anaerobic environment, in particular lacto-fermentation units for the purpose of preserving vegetables, wastewater treatment stations, fish farming ponds with or without temperature control (hot or cold), this brewing system allowing to stir reaction species that are admitted into a reaction volume of the bioreactor, characterised in that this brewing system comprises:
at least one brewing chamber 2, 3, of the flexible and sealed type, each of which is intended to be totally or partly submerged in said reaction volume,
means 4 for receiving a fluid referred to as the filling fluid, the outer volume of each chamber being variable according to the volume of filling fluid admitted into this chamber, and
means 5, 50, 51, 7, 70, 71 for transferring the filling fluid, which are capable of ensuring on the one hand a circulation of the filling fluid, preferably in a closed circuit, between the brewing chambers 2, 3 and the means 4 for receiving the filling fluid, and on the other hand varying the volume of the filling fluid admitted into each brewing chamber.

According to other features of the brewing system according to the invention, which can be taken alone or according to any feature technically compatible for a person skilled in the art:
the filling fluid is a heat transfer liquid, in particular water or an equivalent liquid in terms of aggressivity parameters,
the brewing system comprises at least two brewing chambers 2, 3, and wherein the means 5, 50, 51, 7, 70, 71 for transferring the filling fluid are capable of increasing the volume of the filling fluid in a first chamber 2 while reducing the volume of the filling fluid in a second chamber 3, then reducing the volume of the filling fluid in the first chamber while increasing the volume of the filling fluid in the second chamber,
the flexible and sealed chamber 2,3 comprises an inner skin 25 and an outer skin 26, the outer skin having a high resistance with respect to the aggressive liquids and materials characteristic of the reaction species, while the inner skin has a lower resistance with respect to the filling fluid, which can be water or a heat transfer fluid,
the outer skin 26 is provided with a coating 27 formed by fibres distributed so as to form a flexible mat, capable of favouring the fixing of bacterial biomass in the chamber,
the transfer means comprise, for each brewing chamber, two distinct pipes, one of which 50, 51 allows the arrival of the filling fluid from the receiving means 4 towards said chamber 2, 3, and the other of which 70, 71 allows the exit of the filling fluid from said chamber towards the receiving means,
the receiving means 4 comprise a reservoir 4 for storing said filling fluid, this reservoir being equipped with means for heating this fluid,
the brewing system also comprises means 8, 80 for controlling the temperature of the filling fluid, which are capable of maintaining the temperature of the reaction volume in a suitable range of values,
the maximum outer volume of each brewing chamber is between 1 and 10 cubic metres,
this brewing system further comprises additional control means, which are intended to control the transfer means,
the brewing system further comprises means 16, 17 for fastening each chamber with respect to the walls of a tank of the bioreactor, at the bottom and if necessary at the lateral walls,
the fastening means comprise a rigid frame capable of maintaining reinforced borders, belonging to the flexible chambers, in sealed contact with the bottom and with the walls of the tank, in order to avoid any substantial intrusion of matter or of liquid under the flexible chambers, and
this brewing system further comprises means for injecting a pressurised gas, of the biogas or carbon dioxide type, allowing to ensure micro-oxygenation tangential to the outer surface of each chamber.

The object of the invention is also a bioreactor (I) implemented in particular in milk and cheese factories, fermented dough production units, breweries, wine-making units or even low-temperature fermentation units in an aerobic or anaerobic environment, in particular lacto-fermentation units for the purpose of preserving vegetables, wastewater treatment stations, fish farming ponds with or without temperature control (hot or cold), this bioreactor comprising:
at least one tank 1 defining a reaction volume V1,
means 11 for input of reaction species, which are capable of participating in a reaction of the biochemical type, inside the reaction volume, as well as means 12, 13 for output of products of the reaction,
a brewing system 2, 3 allowing to stir the reaction species that are admitted into the reaction volume, characterised in that this brewing system is as above.

Finally the object of the invention is a method for implementing an above bioreactor, a method in which: reaction species are admitted into the reaction space, filling fluid is admitted into at least one brewing chamber, and a transfer of the filling fluid to this chamber and/or out of this chamber is carried out, so as to vary the outer volume of this chamber and thus brew the reaction species.

According to other features of this method, taken alone or according to any feature technically compatible for a person skilled in the art:
the auxiliary fluid is admitted at a temperature greater than the temperature of the reaction species,
at least two, in particular, three stable values, namely minimum, intermediate and, if necessary, maximum, are assigned to the volume of each chamber.
at least one elementary brewing cycle is carried out comprising a first step in which the volume of the filling fluid in the first chamber is increased, in particular, up to a maximum value, while reducing the volume of the filling fluid in the second chamber, in particular, until a minimum value; and a second step in which the volume of the filling fluid in the first chamber is reduced, in particular, until a minimum value, while increasing the volume of the filling fluid in the second chamber, in particular, until a maximum value, this elementary cycle being repeated if necessary.

### DRAWINGS

The invention will be described below, in reference to the appended drawings, given only as non-limiting examples, in which:

FIG. 1 is a diagram, illustrating the various elements forming a bioreactor that is equipped with a brewing system according to the invention.

FIG. 2 is a diagram, analogous to FIG. 1, illustrating a first step of implementation of the above bioreactor.

FIG. 3 is a diagram, analogous to FIG. 1, illustrating a second step of implementation of the above bioreactor.

FIG. 4 is a diagram, analogous to FIG. 1, illustrating a third step of implementation of the above bioreactor.

FIG. 5 is a diagram, illustrating on a larger scale a part of a flexible chamber belonging to the above brewing system.

### DESCRIPTION

The bioreactor illustrated in the drawings, which is designated as a whole by the reference I, comprises first of all a tank 1. In the example illustrated, this is a single chamber but, as will be shown below, it is possible to use several tanks mutually placed in communication. The wall 10 of this tank defines a reaction volume V1. For this purpose, input means 11 are provided allowing the admission of reaction species, which are capable of participating in a reaction of the biochemical type inside this reaction volume.

Moreover output means 12 allow to evacuate, out of the reaction volume, the substrates digested in a substantially complete manner during the reaction. In a manner known per se, these output means can communicate towards separation means, of the conventional type, which allow to separate a solid fraction and a liquid fraction. Moreover the tank is provided with a roof 13, allowing to collect a gaseous fraction of the reaction, in particular biogas. These structural elements 10 to 13 are not part of the invention, so that they will not be described in more detail below.

In the example illustrated, the wall 10 of the tank defines a single reaction compartment. However, as an alternative not shown, it is possible for this wall to define several compartments. In this respect it is advantageous for the architecture of these compartments to allow the creation and the maintaining of a bioturbation zone. Such an architecture can, for example, be according to the teaching of the French patent 3 045 594 in the name of the applicant.

The bioreactor further comprises a brewing system according to the invention, which allows to stir the reaction species admitted into the reaction volume, according to a swell movement, the frequency and the amplitude of which are variable and controllable. As will be shown below, this brewing system is capable of conferring suitable dynamics onto the desired biochemical reaction.

According to an essential element of the invention, this brewing system comprises first of all at least one brewing chamber, of the flexible and sealed type. The example illustrated uses two distinct chambers 2 and 3 of this type with it being understood that, as will be described in detail below, a different number of chambers can be used.

Flexible and sealed chambers are already known from the prior art, for example from the French Patent Publication No. 2 787 438. In this document each chamber comprises an envelope, which is formed by an inner skin intended to contain water, or an equivalent liquid in terms of aggressivity parameters. Moreover this envelope comprises an outer skin, the resistance of which is clearly higher than that of the inner skin, since this outer skin is intended to come into contact with liquids, the aggressivity of which is significantly greater.

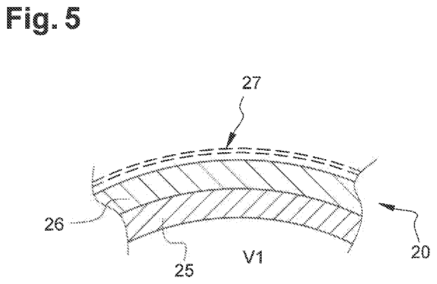

According to the invention, each chamber 2 and 3 has a structure, of which it can be said that it is inverted with respect to the prior art described in the preceding paragraph. This difference is illustrated schematically in FIG. 5, which shows the envelope 20 of the chamber 2, with it being understood that the envelope 30 of the chamber 3 is identical. In substance this envelope 20 is composed of an inner skin 25, which is intended to come in contact with a filling fluid. Advantageously, this filling fluid is a heat transfer liquid, typically water or an equivalent liquid in terms of aggressivity parameters and density. Consequently, this inner skin has a relatively low resistance to aggressions.

However, the outer skin 26 of this envelope 20 is intended to come in contact with much more aggressive fluids, in particular aggressive organic effluents. Consequently, it has a resistance to aggressions that is much greater than that of the inner skin 25. This outer skin 26 is advantageously provided with a coating 27, shown schematically, which is formed by fibres referred to as free and short, the rigidity of which is variable. These fibres are distributed so as to form a flexible mat, capable of favouring the fixing of bacterial biomass in the tank.

Besides the above difference, the manufacturing of these chambers 2 and 3 is substantially identical to that of the chambers of the prior art, in particular described in the French patent above. The envelopes 20 and 30 are typically composed of special cloths made of woven plastic fibres, generally coated with PVC polyvinyl chloride. These envelopes are assembled in a manner known per se, by any suitable means, in particular by being welded and sewed.

These chambers have mechanical features, which are capable of making them resistant to high external pressures. In this respect, they can in particular resist stresses exerted by agricultural machines. As non-limiting examples, they can have a resistance to a water-gauge pressure of 8 m maximum, or 0.8 bar. Besides this mechanical resistance, these envelopes are capable of resisting over a long term relatively high temperatures, which are those occurring in the context of the implementation of methane digesters.

These chambers can adopt a wide range of geometric alternatives, which confers onto them a possibility of adaptation in tanks having different architectures. For example mention will be made of an orthogonal geometry, of the type square, rectangle or right-angled triangle. Mention will also be made of a geometry in the shape of a disc, or of a portion of a disc.

Because of their flexible nature, these chambers 2 and 3 are capable of adopting, in a stable manner, different sizes. When they are inflated in a maximum manner, their size can be between 1 and 10 cubic metres. The limitation of volume is more particularly linked to the flow rates of the pumps that supply them, which must be relatively high to ensure the swell movement required for effective brewing. Moreover, at least one other configuration of a smaller size can be provided. In the example illustrated, each chamber is capable of having a size referred to as minimum which corresponds for example to approximately one third of the maximum size, as well as a size referred to as intermediate which corresponds for example to approximately two thirds of the maximum size.

Beyond the constraint of dimensioning of the pumps that serve these chambers, the dimensional parameterisation of the chambers depends substantially on the volume of the tanks, their diameter, the tank-bottom surface, but especially on the height of the column of digestates in the tanks. These constraints fall under the technical feasibility of the method, so that the most sensible and the least costly solution for getting around these constraints is to prefer inflatable chambers of a small size disposed side by side. In this respect, the maximum dimension of each chamber does not exceed a voltage of approximately 10 m³.

Advantageously, each chamber is fastened onto the tank 10. Preferably, there is a system for fastening onto the bottom 14 of the tank. This fastening system, which is schematically illustrated while being assigned the reference 16, is of any suitable type. In this respect, it can for example be chosen to use a tubular frame anchored at the tank bottom by bolts that pass through the peripheral margin of the chambers. Moreover, it is also possible to use a similar fastening system but installed on the lateral wall 15 of this tank. The fastening of the chamber, with respect to the tank, allows to avoid the possible flotation of this chamber. This phenomenon can in particular occur when the weight of the chamber, inflated with the filling liquid, is less than that of the reaction species contained in the digester. This fastening further allows to deploy the chamber in a satisfactory manner, during the various implementation phases, while preventing the interference of digestates between the tank bottom and the brewing chambers.

The brewing system according to the invention further comprises a drum 4, allowing the reception of the filling liquid, intended to supply the chambers 2 and 3. This drum 4 is placed under heat exchange, by an exchanger 40 of any suitable type, with a reserve 41 containing a heat transfer fluid, typically hot water. For this purpose a line 42, equipped with a solenoid valve 43, extends between this reserve 41 and this exchanger 40. Downstream of this exchanger, an additional line 44 ensures the recycling of all or part of the heat transfer fluid, in the direction of the reserve 41. This reserve is of any suitable type, known per se: mention will be made in a non-limiting manner of the heating tank of a bain-marie, a boiler supplied with biomethane or biogas, a solar thermal or mixed photovoltaic and thermal plant, a passive system for recovering the heat on the walls of a composting silo in a thermophilic zone, or any combination of the devices listed immediately above.

The brewing system according to the invention further comprises means for transferring the filling liquid, mentioned above. The drum 4 is first of all placed in communication with each chamber, by this filling liquid. For this purpose, a main intake line 5 extending immediately downstream of the drum, which opens into branch intake lines 50 and 51, each of which is connected to the inner volume of a respective chamber, is provided. The main line 5 is equipped with a temperature sensor 52 of any suitable type, as well as with a flowmeter 53. Moreover, each branch line is equipped with a respective solenoid valve 54, 55.

A pump 6 is further provided, allowing to move the filling liquid along the lines 5, 50 and 51. Advantageously this pump 6 is capable of moving the filling liquid which can reach a temperature of 60° C. with a parameterisation of barometric pressure adapted to the configuration of the flexible chambers on the following basis (without inferring head losses):
Dp=(Vi−Vm)/T, where Dp is the nominal flow rate of the pump given in m³/h, Vi is the intermediate volume of a flexible cistern, Vm is the minimum volume of a flexible cistern and T the duration expressed in hours chosen to go from the state Vm to the state Vi, and
Pp=((ρ·Hs)/10)·1.25, where Pp is the nominal service pressure of the given pump in bar (10 m water-gauge) (with a prudence factor of 1.25) that the pump must deliver, ρ is the density of the digestates, Hs the height of the column of digestates above the surface of the submerged inflatable drums in the stage Vm.

Moreover, various pipes allow the return of the filling liquid, from each chamber 2 and 3 towards the drum 4. For this purpose, two branch lines referred to as return are connected onto the envelopes of the chambers. These branch lines 70 71 open into a return line referred to as main, which is placed in communication with the drum 4. Each branch line 70 71 is equipped with a solenoid valve 72 73, as well as with a decompression valve 74 75. Moreover, the main line 7 is equipped with a flowmeter 76, as well as with a solenoid valve 77.

As shown above, the various branch lines 50,51 70 71 are connected onto the envelopes of the brewing chambers. The mechanical connection of these lines, at these envelopes, is carried out in a manner known per se. For example, the solution described in the aforementioned French Patent Publication No. 2 787 438 can be used.

The parameterisation of the pump is also dependent on the maximum and minimum volume of the chambers which determines the amplitude of their variation in volume and on the frequency of this variation which is expressed in terms of flow rate. Thus for a chamber of 1 m³ maximum filling and 0.25 m³ minimum filling, or a total theoretical amplitude of 0.75 m³ with a frequency of 30 s or 0.03 Hz the flow rate of the pump should be calculated as follows.

The real amplitude to be taken into account is (0.75 m³+0.25 m³)/2 since when the filling liquid of the first chamber empties into the second chamber under the effect of the pressure of the reaction species contained in the bioreactor the equilibrium is reached when each chamber contains 0.5 m³. The pump must therefore mobilise 0.25 m³ to bring the filling of the second chamber to 0.75 m³. The placement into equilibrium via gravity, without pumping can take 10 seconds and if it is desired for the transfer to take 30 seconds. The pump must therefore mobilise 0.25 m³ in 20 seconds or 45 m³/h. The pumping can also occur during the phase of gravitational balancing which substantially shortens the duration of this phase.

Finally the brewing system according to the invention advantageously comprises control means, which include first of all a control unit 8, shown schematically. This unit is first of all in communication, via a line 81, with a temperature sensor 80, which is submerged in the reaction volume of the tank. Moreover, this control unit 8 is connected, via respective lines 82 and 83, to the flowmeters described above. Finally this unit 8 is capable of controlling the various solenoid valves described above, via control lines that are not shown.

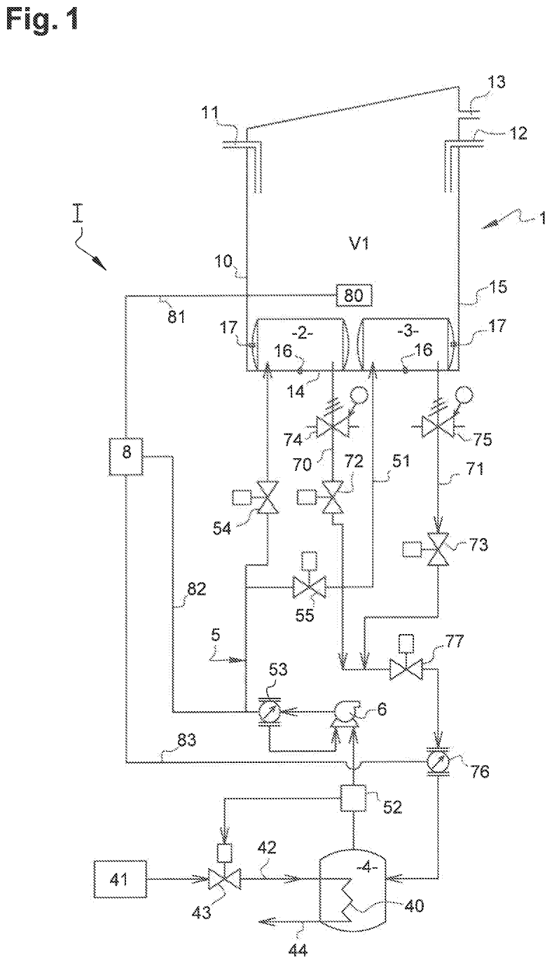

An example of implementation of the bioreactor 1 equipped with a brewing system according to the invention, as described above, will now be presented, in particular in reference to FIGS. 1 to 4.

The reaction species are first admitted into the reaction space, via the pipe 11. Moreover, each chamber 2 and 3 is filled via filling liquid, via the successive lines 5, then 50 and 51. The instant at which the initial filling is carried out is globally not important, since this is a system with continuous feeding.

First it is supposed that, as shown in FIG. 1, each chamber is filled approximately halfway, so as to adopt the intermediate configuration presented above. It is then supposed that, according to the invention, it is desired to carry out a brewing of the reaction species initially admitted into the inner volume V1. The brewing is sequenced in amplitude and in frequency in a robot, and a brewing session can last up to 20 minutes for example.

For this purpose, this means varying the respective sizes of the two chambers. The transfer means, belonging to the brewing system of the invention, are capable of varying the volumes of filling fluid in the two chambers. Advantageously this volume increases in the first chamber while it decreases in the second chamber, and vice versa.

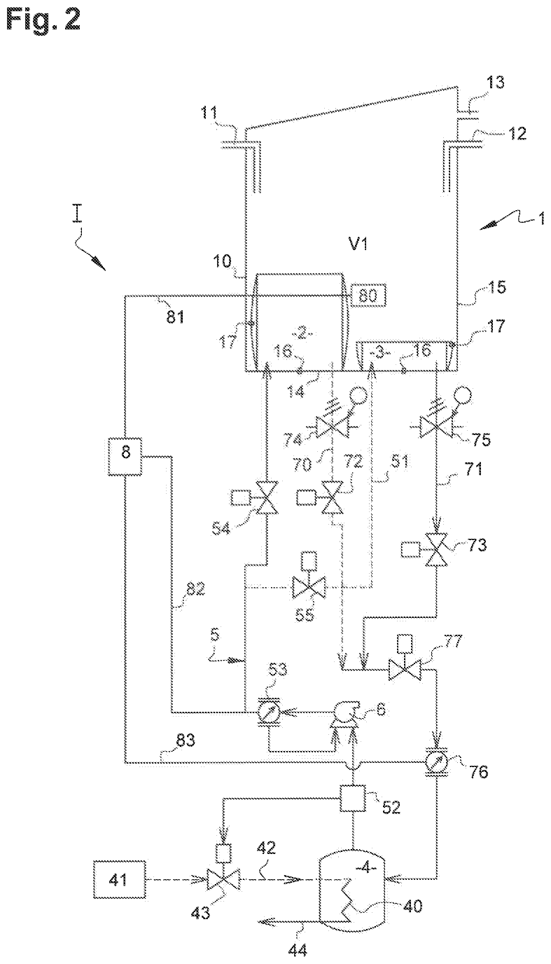

In substance, as shown in FIG. 2, the solenoid valves respectively 55 and 72 are closed. This allows to prohibit the flow of filling liquid, on the one hand towards the inside of the chamber 3, on the other hand towards the outside of the channel 2. It is understood that, in these conditions, the chamber 2 tends to fill up, until it adopts its maximum size. First, this chamber reaches a phase of equilibrium with the other chamber, via gravity, then continues to fill up under the effect of the pump. Moreover, the chamber 3 tends to empty itself, until it adopts its minimum size.

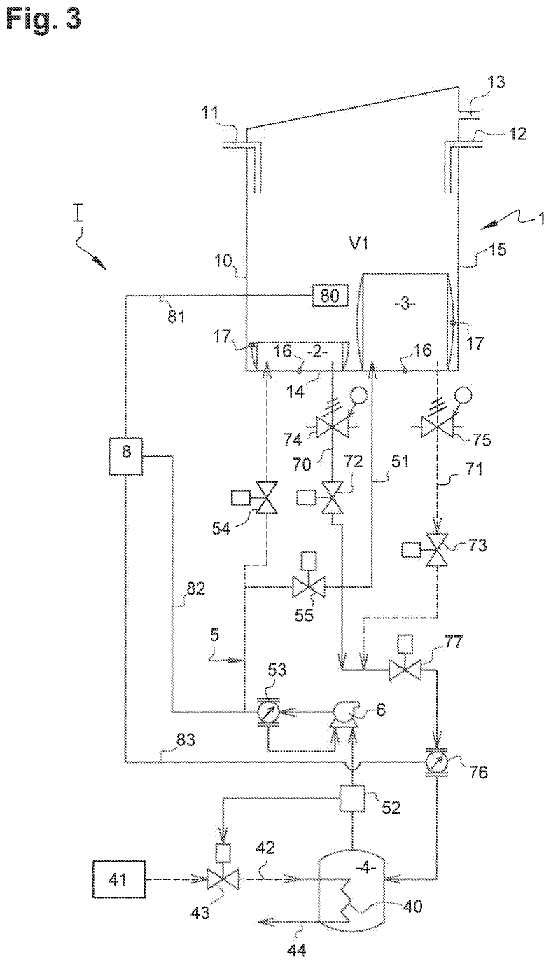

Then, the reverse operation is carried out, namely the solenoid valves 55 and 72 are once again opened while, according to the sequence programmed in the robot, the solenoid valves 54 and 73 are closed. This thus allows to prohibit the flow of filling liquid both into the chamber 2 and out of the chamber 3 while allowing this flow into the chamber 3 and out of the chamber 2. In these conditions, the chamber 2 tends to empty itself, until it adopts its minimum size, while the chamber 3 tends to fill up, until it adopts its maximum size. This configuration is illustrated in FIG. 3.

It is possible to carry out a repetitive implementation of the elementary cycle described in detail above, namely to make the chambers 2 and 3 go several times in an alternating manner successively into their configurations of FIGS. 2 and 3. These phases of deployment and of retraction of the chambers confers a brewing effect referred to as pulsed, at the bottom of the tank. This induces a positive displacement, also ensuring the thermal transfer with a weak gradient of the digestates received in this tank.

The use of the inflatable chambers, according to the invention, is very particularly advantageous. Indeed, the brewing thus carried out is substantially non-destructive to the bacterial biomes. Moreover, it is likely to advantageously disturb, both upward and downward in incident curved fallout, the digestates over the entire column. This guarantees the desired swell effect at tank bottom, with effects being transferred throughout the tank. This swell effect is very particularly significant when at least two brewing chambers are used. It is noted that such an effect cannot be obtained, by implementing the teaching of the Chinese document presented above, given that the latter uses a single chamber.

Finally a transfer of thermal energy is advantageously carried out, according to the invention, via the necessary rise in temperature of the reaction species. This thermal transfer can be controlled, via the control unit. According to the temperature measured by the sensor submerged in the tank, the control unit is capable of controlling the various solenoid valves, so as to make the filling liquid transit into a heat exchanger, inside of which it will be loaded with calories to meet the needs for transfer of these calories in the bioreactor.

In this respect, the use of a heat transfer liquid as filling fluid allows to ensure two functions simultaneously in an efficient manner, namely the brewing but also the thermal transfer. It is noted that the teaching of the Chinese document above does not allow to carry out these combined functions in a satisfactory manner, given that the filling fluid used is air. However, the latter clearly has worse performance than a liquid, in terms of thermal transfer.

With regard to the Chinese document, it is also emphasised that it does not mention important factors, which are advantageously taken into account in the present patent application. On the one hand the aggressivity of the digestates for the outer surface of the envelopes, on the other hand the impermeability to water for their inner face, constitute limiting parameters, particularly critical when the volume of the envelope must be varied. However, such parameters are not clearly addressed in this Chinese document.

According to the invention, the combination of the addition of organic substrates rich in digestible carbon, the dilution of the entering substrates carried out with solutions rich in intensifiers of the bacterial metabolism and this pulsed brewing which also produces a very efficient inertial thermal regulation favours the maintaining of a bioturbation zone in a tank of the digester.

Indeed, the solid carbon matter, the raw compost, has a low density that maintains it at the surface, the solutes for dilution and for metabolic intensification, the composting percolates close to the density of water are present throughout the volume of the tank while the denser mineral coenzymes rapidly migrate to tank bottoms.

The pulsed brewing creates a swell movement, the frequency of which is low, but the amplitude of which is significant and which is weakly turbulent. This swell movement, obtained according to the invention, is particularly effective since it adopts the form of a positive-displacement flow coming from the middle of the tank, with emergence of a digestate lens at the centre of the surface of the tank. This leads to the hydraulic collapse of the digestates floating at the periphery and towards the bottom, without the densest fraction being driven towards the top of the tanks but rather maintained at the bottom regardless of the movement of the digestates. These dynamics prevent in particular the formation of a stable floating crust at the surface of the tanks. They further maintain a certain fluidity as well as a reduced coalescence at tank bottom, which is advantageous with a view to a regular extraction by pumping of undesirable sediments.

Indeed the dense and solid matter with a small particle size but non-colloidal rapidly constitutes at tank bottom a fixing base for dominant bacterial colonies and their symbiotes, commensals and competitors. The minerals, bones, nails, beaks, cartilage, but also bark or fragments of wood form most of these fixing grains. The slow brewing maintains dynamics of exchange in this zone rich in fixed biomass, the bioturbation effect is thus perfectly ensured.

This tank bottom architecture is certainly favourable to the fixing of active biomass. However, through accumulation, it tends to reduce the reaction volume in the bioreactor. It must consequently be advantageously regulated, via regular extractions.

The method for implementing the brewing system and the bioreactor that are according to the invention has a remarkable effectiveness, which is based on major factors of maintaining and activating bioturbation mechanisms. The latter comprise the mechanism of biochemical enrichment by partial recycling in the digester of the composts and composting percolates, the maintaining of the tank at optimal temperature with an inertial vector with a weak thermal gradient which also ensures the maintaining at a temperature relatively higher in the bioturbation zone with respect to the rest of the column, as well as the pulsed brewing combined with micro-oxygenation by recirculation of the biogas purified of H₂S.

The reaction process occurring in the tank can be of any suitable type, namely of the infinitely mixed or multiphase type, with continuous or sequential loading, with free or fixed biomass, mesophilic or thermophilic. Moreover, the invention can be applied to reactions involving all possible ranges of concentration of volatile organic solids.

As the reaction takes place, the biogas produced is extracted via the roof 13. For reasons of economy and simplification of the automation of the brewing process, a sharing of the heating, pumping, flow rate regulation and thermal transfer means is implemented. In substance, each bioreactor tank receives, as dedicated equipment, only pipes and solenoid valves. This induces that the brewing carried out in a bioreactor tank is organised in a session with a limited duration. In this respect, a duration of 20 minutes is a good period for methane digesters including 3 bioreactor tanks. Indeed, this allows to leave a tank without brewing with swell effect for approximately 40 minutes, which is a time interval easily tolerable on the biochemical level, in particular if oxygenation is maintained during this period.

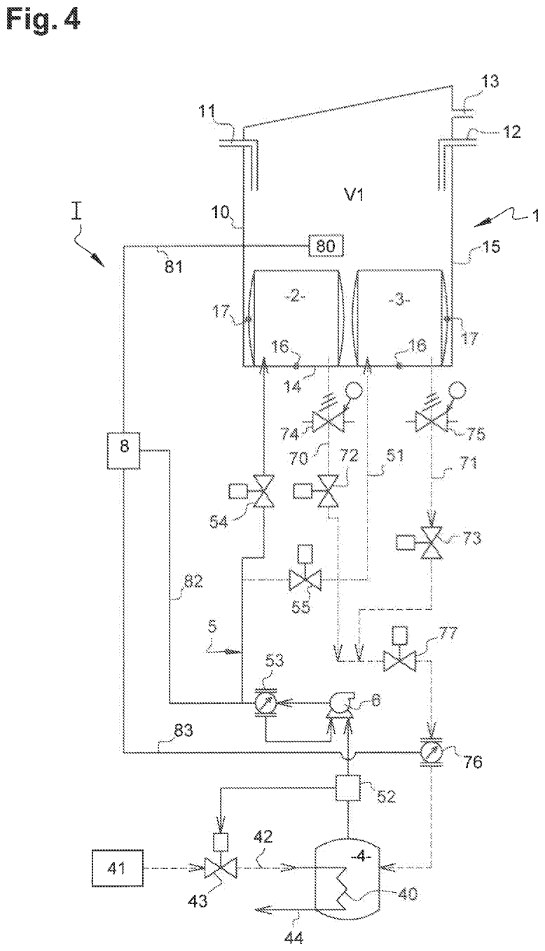

Advantageously, the brewing chambers can be used in their configuration referred to as overflowing. For this purpose, as shown in FIG. 4, the 2 chambers 2 and 3 are filled, so that they adopt at the same time their maximum size. It is thus understood that, in these conditions, the sum of the volumes occupied by the chambers and the substrates becomes greater than the volume of the tank. Consequently, a part of the substrates is evacuated by the lines 12, so as to be separated into a liquid fraction and a solid fraction.

A person skilled in the art can vary in various manners the size of the chambers 2 and 3, according to the time. According to a first possibility not shown, the brewing sequence can comprise several alternating phases of filling then of emptying, for each chamber. Of course other types of variations can be provided, according to that which is desired by a person skilled in the art.

In the example illustrated, for reasons of dynamics of the fluids and thermodynamics, 2 independent inflatable chambers are used inside the same tank. However, as an alternative, for example in a multiphase digester, it is possible to install a single chamber per tank, in particular if the tank considered corresponds strictly to one phase and if its size is sufficiently small. In the latter case, it is possible for example to provide several tanks disposed next to each other, operating via overflowing.

In general, a succession of two to three inflatable drums of a small size disposed side by side is preferable to any other arrangement. In this configuration the brewing is thus carried out without it being necessary to have available an adiabatic reserve of hot water of a significant volume at the periphery of the digester to manage the flows and refluxes of the heat transfer fluid. Indeed the volume extracted in a drum is transferred into a neighbouring drum, and so on, so as to create a swell wave at tank bottom as described in more detail above. The outer drum is mobilised to inject a volume of heat transfer fluid, which is incremented above the medium volume, only if it is desired to obtain an overflow effect.

The implementation of this system for non-destructive transfer, brewing and overflow of the digestates also has the advantage of being completed by a system for injection of biogas purified of H₂S, or of CO₂ under a pressure sufficient to ensure micro-oxidation tangentially to the upper surface of the inflatable drums.

The brewing system according to the invention, a bioreactor equipped with this brewing system, as well as its implementation method, can be associated with additional structural and functional elements. The latter, which should be taken into account for the correct operation of this bioreactor, are not however part of the invention. They will not therefore be described in more detail. These elements are in particular:
solenoid valves, pumps, flowmeters, volume and temperature sensors, drums and additional heat exchangers,
a system for grinding the entering waste and substrates that allows to reduce their relative size into a particle size not exceeding 25 mm. This device can take the form of a dual-axis slow knife mill served by a loading hopper ensuring the protection of the operator,
a preheating and mixing system consisting of a bain-marie or any other equivalent device loaded gravitationally with the organic substrates and the raw compost coming from the mill and receiving the dilution liquid consisting of percolates,
a lift pump accepting highly turbid flows with a maximum particle size of 35 mm intended to supply the bioreactor in the upper part of the latter,
a network of sensors that measure in real or slightly deferred time the values obtained for the temperature, the pH, the turbidity of the digestates during the various phases, the chemical composition, the temperature and the relative humidity of the biogas and of the purified biomethane,
a set of several programmable industrial robots that process the signals received from the sensors, infer the behaviour of effectors and report on the state of the system on a remote control station,
a network of effectors such as hydraulic or pneumatic solenoid valves that regulate the circulation of the flows of substrates, digestates, eluates controlled by the programmable robots or directly by the human operator,
one or more tanks referred to as auxiliary, distinct from the tank 1 described in the drawings, which act as bioreactors to house the various phases of the biodigestion with the means for introducing and evacuating the treated matter,
a system for degassing the digestates at the end of the methane digestion cycle and which can be a simple decantation chamber impermeable to the gas provided or not with specific brewing devices,
a device for filtering the biogas, with the function of separating and treating CO₂ and CH₄ and which can take the form of a cell for solubilisation in water, solvents, reactants, osmotic filters or any other equivalent device,
a device for dehumidification of the biogas having the function of extracting the H₂O water by condensation, and
a device for filtering the biogas having the function of separating and treating the sulphurated hydrogen (H₂S), the siloxanes and nitrogen oxides and which can take the form of a cell for capturing biologically, with activated carbon, or any other equivalent device.

1-18. (canceled)

19. A brewing system, comprising:
at least one flexible and sealed brewing chamber, each brewing chamber operable to be totally or partially submerged in a reaction volume of a bioreactor;
a filling fluid chamber to receive a filling fluid in a manner such that an outer volume of each brewing chamber is variable according to the volume of filling fluid admitted into the filling fluid chamber; and
a closed fluid circuit to transfer and circulation of the filling fluid between the brewing chambers and the filling fluid chamber, the closed fluid circuit being operable to vary the volume of the filling fluid admitted into each brewing chamber.

20. The brewing system of claim 19, wherein the filling fluid comprises a heat transfer liquid or water.

21. The brewing system of claim 19, wherein the closed fluid circuit is operable to increase the volume of the filling fluid in a first brewing chamber of the at least one flexible and sealed brewing chamber, while reducing the volume of the filling fluid in a second brewing chamber of the at least one flexible and sealed brewing chamber, then reduce the volume of the filling fluid in the first brewing chamber while increasing the volume of the filling fluid in the second brewing chamber.

22. The brewing system of claim 19, wherein each brewing chamber in the at least one flexible and sealed brewing chamber comprises an inner skin and an outer skin, the outer skin having a high resistance with respect to aggressive liquids and materials characteristic of a reaction species, while the inner skin has a lower resistance with respect to the filling fluid.

23. The brewing system of claim 22, wherein the outer skin is provided with a coating formed by fibres distributed to form a flexible mat that is operable to promote a fixing of bacterial biomass in the brewing chamber.

24. The brewing system of claim 19, wherein the closed fluid circuit comprises, for each brewing chamber:
a first pipe which allows flow of the filling fluid from the filling fluid chamber towards the at least one flexible and sealed brewing chamber, and
a second pipe which allows an exit of the filling fluid from the at least one flexible and sealed brewing chamber towards the filling fluid chamber.

25. The brewing system of claim 19, wherein:
the filling fluid chamber comprises a reservoir for storing the filling fluid, and
the reservoir includes a heater for heating the filling fluid.

26. The brewing system of claim 19, further comprising a temperature control unit operable to control a temperature of the filling fluid and maintaining a temperature of the reaction volume in a suitable range of temperature values.

27. The brewing system of claim 19, wherein each brewing chamber has a maximum outer volume of between 1 and 10 cubic metres.

28. The brewing system of claim 19, wherein further comprising a fluid circuit control unit, operable to control the closed fluid circuit.

29. The brewing system of claim 19, wherein further comprising a fastening device operable to fasten each brewing chamber with respect to walls of a tank of the bioreactor and a bottom thereof.

30. The brewing system of claim 29, wherein the fastening device comprises a rigid frame operable to maintain reinforced borders in sealed contact with the bottom and the walls of the tank to prevent any substantial intrusion of matter or of liquid under the brewing chamber.

31. The brewing system of claim 19, further comprising a gas injector operable to inject a pressurised gas, of a biogas or carbon dioxide type, to ensure micro-oxygenation tangential to an outer surface of each brewing chamber.

32. The brewing system of claim 19, wherein the brewing system is arranged in a bioreactor implemented in milk and cheese factories, fermented dough production units, breweries, wine-making units, low-temperature fermentation units in an aerobic or anaerobic environment, lacto-fermentation units for preserving vegetables, wastewater treatment stations, and fish farming ponds with or without temperature control.

33. The brewing system of claim 32, wherein the brewing system is operable to allow stirring of reaction species that are admitted into a reaction volume of the bioreactor.

34. A bioreactor, comprising:
at least one tank defining a reaction volume;
an input device operable to input a reaction species which are capable of participating in a biochemical reaction inside the reaction volume;
an output device operable to output products of the biochemical reaction; and
a brewing system operable to stir the reaction species that are admitted into the reaction volume, the brewing system including:
at least one flexible and sealed brewing chamber, each brewing chamber operable to be totally or partially submerged in the reaction volume;
a filling fluid chamber to receive a filling fluid in a manner such that an outer volume of each brewing chamber is variable according to the volume of filling fluid admitted into the filling fluid chamber; and
a closed fluid circuit to transfer and circulation of the filling fluid between the brewing chambers and the filling fluid chamber, the closed fluid circuit being operable to vary the volume of the filling fluid admitted into each brewing chamber.

35. A method for operating a bioreactor of claim 34, the method comprising:
admitting the reaction species into the reaction volume;
admitting the filling fluid into at least one brewing chamber;
conducting a transfer of the filling fluid towards the at least one brewing chamber and/or out of the at least one brewing chamber, to vary an outer volume of the at least one brewing chamber and thereby brew the reaction species.

36. The method of claim 35, wherein the filling fluid is admitted at a temperature greater than a temperature of the reaction species.

37. The method of claim 35, wherein at least three stable values, including a minimum value, an intermediate value, and a maximum value, are assigned to the volume of each brewing chamber.

38. The method of claim 35, further comprising:
conducting at least one elementary brewing cycle by:
increasing a volume of the filling fluid in a first brewing chamber up to a maximum value, while reducing a volume of the filling fluid in a second brewing chamber until a minimum value;
reducing the volume of the filling fluid in the first brewing chamber until a minimum value, while increasing the volume of the filling fluid in the second brewing chamber up to a maximum value; and
repeating at least one elementary cycle when necessary.
